(19) United States
(12) Patent Application Publication
(10) Pub. No.: US 2021/0369790 A1
(43) Pub. Date: Dec. 2, 2021

(54) METHODS AND MATERIALS FOR IDENTIFYING AND TREATING MAMMALS RESPONSIVE TO OBESITY TREATMENTS

(71) Applicant: Mayo Foundation for Medical Education and Research, Rochester, MN (US)

(72) Inventors: Michael D. Jensen, Rochester, MN (US); Vandana Nehra, Rochester, MN (US); Purna C. Kashyap, Rochester, MN (US)

(73) Assignee: Mayo Foundation for Medical Education and Research, Rochester, MN (US)

(21) Appl. No.: 16/756,458

(22) PCT Filed: Sep. 16, 2018
(86) PCT No.: PCT/US2018/056109

§ 371 Date: Apr. 15, 2020

Related U.S. Application Data
(60) Provisional application No. 62/573,100, filed on Oct. 16, 2017.

(51) Int. Cl. A61K 35/74; A61K 45/06; A61P 3/04

(52) U.S. Cl. CPC A61K 35/74; A61P 3/04; A61K 45/06

(57) ABSTRACT

This document provides methods and materials for identifying and treating mammals responsive to obesity treatments. For example, methods and materials for assessing a mammal's gut microbiota (e.g., a human's gut microbiota) to identify that mammal (e.g., human) as being responsive to an obesity treatment are provided. Methods and materials for treating obesity by assessing a mammal's gut microbiota (e.g., a human's gut microbiota) to identify that mammal (e.g., human) as being responsive to an obesity treatment and proceeding with an obesity treatment also are provided.

### CROSS-REFERENCE TO RELATED APPLICATIONS

This application claims priority to U.S. Application Ser. No. 62/573,100, filed on Oct. 16, 2017. The disclosure of the prior application is considered part of the disclosure of this application, and is incorporated in its entirety into this application.

### BACKGROUND

### 1. Technical Field

This document relates to methods and materials involved in identifying and treating mammals responsive to obesity treatments. For example, this document provides methods and materials for assessing a mammal's gut microbiota (e.g., a human's gut microbiota) to identify that mammal (e.g., human) as being responsive to an obesity treatment (e.g., a nutritional intervention, a physical activity intervention, and/or a comprehensive lifestyle intervention program). This document also provides methods and materials for treating obesity by assessing a mammal's gut microbiota (e.g., a human's gut microbiota) to identify that mammal (e.g., human) as being responsive to an obesity treatment (e.g., a nutritional intervention, a physical activity intervention, and/or a comprehensive lifestyle intervention program) and proceeding with an obesity treatment (e.g., a nutritional intervention, a physical activity intervention, and/or a comprehensive lifestyle intervention program).

### 2. Background Information

Obesity is a chronic disease that is increasing in prevalence around the world and is now considered a global epidemic. Obesity, as measured by body mass index (BMI) of 30 kg/m² or greater, has been consistently associated with increased all-cause mortality. A comprehensive lifestyle intervention is usually the first step for achieving weight loss. A weight loss of just 5 percent through a combination of dietary restrictions, physical exercise, and behavioral therapy is effective in achieving better glycemic control and preventing diabetes. However, there is marked inter-individual variability in the success of this approach that has often been attributed to patient compliance.

### SUMMARY

This document provides methods and materials for identifying and treating mammals responsive to obesity treatments. For example, this document provides methods and materials for assessing a mammal's gut microbiota (e.g., a human's gut microbiota) to identify that mammal (e.g., human) as being responsive to an obesity treatment. This document also provides methods and materials for treating obesity by assessing a mammal's gut microbiota (e.g., a human's gut microbiota) to identify that mammal (e.g., human) as being responsive to an obesity treatment and proceeding with an obesity treatment.

As described herein, humans responsive to a weight loss treatment (e.g., a comprehensive lifestyle intervention program) such that they are capable of losing at least about 5 percent of their body weight can be identified based on particular gut microbiota (e.g., elevated levels of Phascolarctobacterium, reduced levels of Veillonella, and/or reduced levels of Dialister). In some cases, mammals (e.g., humans) having particular gut microbiota (e.g., reduced levels of Phascolarctobacterium, elevated levels of Veillonella, and/or elevated levels of Dialister) can be identified as not being responsive to obesity treatments such as nutritional interventions, physical activity interventions, and/or comprehensive lifestyle intervention programs.

In one aspect, this document features a method for identifying a mammal as being responsive to an obesity or over weight treatment. The method can include (a) determining that the mammal has an elevated level of Phascolarctobacterium within the mammal's gut microbiota, a reduced level of Veillonella within the mammal's gut microbiota, or a reduced level of Dialister within the mammal's gut microbiota, and (b) classifying the mammal as being responsive to an obesity or over weight treatment. The mammal can be a human. The treatment can be a diet or physical exercise.

In another aspect, this document features a method for identifying a mammal as not being responsive to an obesity or over weight treatment. The method can include (a) determining that the mammal has a reduced level of Phascolarctobacterium within the mammal's gut microbiota, an elevated level of Veillonella within the mammal's gut microbiota, or an elevated level of Dialister within the mammal's gut microbiota, and (b) classifying the mammal as not being responsive to an obesity or over weight treatment. The mammal can be a human. The treatment can be a diet or physical exercise.

In another aspect, this document features a method for treating an overweight or obese mammal. The method can include (a) identifying the mammal as having an elevated level of Phascolarctobacterium within the mammal's gut microbiota, a reduced level of Veillonella within the mammal's gut microbiota, or a reduced level of Dialister within the mammal's gut microbiota, and (b) treating the mammal with an obesity or over weight treatment. The mammal can be a human. The treatment can be a diet or physical exercise.

In another aspect, this document features a method for treating an overweight or obese mammal. The method can include treating a mammal identified as having an elevated level of Phascolarctobacterium within the mammal's gut microbiota, a reduced level of Veillonella within the mammal's gut microbiota, or a reduced level of Dialister within the mammal's gut microbiota with an obesity or over weight treatment. The mammal can be a human. The treatment can be a diet or physical exercise.

In another aspect, this document features a method for increasing the effectiveness of a weight loss treatment. The method can include administering a composition containing live Phascolarctobacterium to a mammal, wherein the level of Phascolarctobacterium within the mammal is increased. The mammal can be a human. The weight loss treatment can be a diet or physical exercise. In some cases, over 90 percent, over 95 percent, or all of the live bacteria of the composition can be Phascolarctobacterium.

This document also features a method for increasing the effectiveness of a weight loss treatment. The method can include administering, to a mammal, a composition containing a microbiome modifying agent having the ability to reduce the number of Veillonella organisms or Dialister organisms within the mammal, wherein the number of Veillonella organisms or Dialister organisms within the mammal is reduced. The mammal can be a human. The weight loss treatment can be a diet or physical exercise. In some cases, the microbiome modifying agent can be antibiotics.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Linear discriminant analysis effect size analysis results showing baseline compositional and functional features that are predictive of at least 5% weight loss (success=light color) or less than 5% weight loss (failure=darker color) after 3 months. A, Cladogram showing the location of the Veillonellaceae family containing Phascolarctobacterium (darker color) and Dialister (lighter color) within the phylogenetic tree. B, LDA scores for the two identified bacterial biomarkers of weight loss. C, Increased abundance of a transposase (COG3328) predicts at least 5% weight loss. D, Increased abundance of six carbohydrate-active enzymes and binding proteins were predictive of losing less than 5% of baseline weight. LDA=linear discriminant analysis.

### DETAILED DESCRIPTION

This document provides methods and materials for identifying and treating mammals responsive to obesity and/or weight loss treatments. For example, this document provides methods and materials for assessing a mammal's gut microbiota (e.g., a human's gut microbiota) to identify that mammal (e.g., human) as being responsive to an obesity and/or weight loss treatment. This document also provides methods and materials for treating obesity by assessing a mammal's gut microbiota (e.g., a human's gut microbiota) to identify that mammal (e.g., human) as being responsive to an obesity and/or weight loss treatment and proceeding with an obesity and/or weight loss treatment. Examples of obesity and/or weight loss treatments include, without limitation, nutritional interventions (e.g., diets), physical activity intervention (e.g., exercise), and comprehensive lifestyle intervention programs such as those that combine nutritional interventions and physical activity interventions.

As described herein, a mammal (e.g., a human) can be identified as being responsive to an obesity and/or weight loss treatment based on the presence of an elevated level of Phascolarctobacterium within the mammal's gut microbiota, a reduced level of Veillonella within the mammal's gut microbiota, and/or a reduced level of Dialister within the mammal's gut microbiota. In some cases, a mammal (e.g., a human) can be identified as not being responsive to an obesity and/or weight loss treatment based on the presence of a reduced level of Phascolarctobacterium within the mammal's gut microbiota, an elevated level of Veillonella within the mammal's gut microbiota, and/or an elevated level of Dialister within the mammal's gut microbiota.

Any appropriate method can be used to determine the levels of particular microorganisms present within a mammal's gut microbiota. For example, the methods described herein can be used to determine the levels of particular microorganisms present within a mammal's gut microbiota.

Once a mammal is identified as being responsive to an obesity and/or weight loss treatment as described herein, that mammal can be treated with an obesity and/or weight loss treatment. For example, a human (e.g., overweight or obese human) identified as having an elevated level of gut Phascolarctobacterium, a reduced level of gut Veillonella, and/or a reduced level of gut Dialister can be treated with an obesity and/or weight loss treatment (e.g., a diet).

In some cases, the presence of an elevated level of transposase (e.g., COG3328) expression within a microbiota sample can be used alone or in combination with an elevated level of gut Phascolarctobacterium, a reduced level of gut Veillonella, and/or a reduced level of gut Dialister to identify a mammal (e.g., an obese or over weight human) as being responsive to an obesity and/or weight loss treatment as described herein. In some cases, the presence of an elevated level of expression of one or more carbohydrate-active enzymes or binding proteins within a microbiota sample can be used alone or in combination with an reduced level of gut Phascolarctobacterium, an elevate level of gut Veillonella, and/or an elevated level of gut Dialister to identify a mammal (e.g., an obese or over weight human) as not being responsive to an obesity and/or weight loss treatment as described herein. Examples of such carbohydrate-active enzymes or binding proteins that can be detected within a microbiota sample and used to identify a mammal (e.g., an obese or over weight human) as not being responsive to an obesity and/or weight loss treatment include, without limitation, glycoside hydrolases (e.g., GH67 and GH93), carbohydrate-binding module family members (e.g., CBM51 and CBM58), carbohydrate esterases (e.g., CE6), and glycosyltransferases (e.g., GT25).

In some embodiments, the elevated levels of gut bacteria (e.g., Phascolarctobacterium) and the reduced levels of gut bacteria (e.g., Dialister, or Veillonella) can be determined in comparison to the corresponding levels present in the population that did not lose at least 5% body weight after the lifestyle intervention program described herein. The reduced levels of gut bacteria (e.g., Phascolarctobacterium) and the elevated levels of gut bacteria (e.g., Dialister, or Veillonella) can be determined in comparison to the corresponding levels present in the population that lost at least 5% body weight after the lifestyle intervention program described herein. The elevated levels of carbohydrate-active enzymes or binding proteins (e.g., GH67, GH93, CBM51, CBM58, CE6, or GT25) can be determined in comparison to the corresponding levels present in the population that lost at least 5% body weight after the lifestyle intervention program described herein. The reduced levels of carbohydrate-active enzymes or binding proteins (e.g., GH67, GH93, CBM51, CBM58, CE6, or GT25) can be determined in comparison to the corresponding levels present in the population that did not lose at least 5% body weight after the lifestyle intervention program described herein.

In some cases, an obese or over weight mammal such as an obese or over weight human having a gut microbiota that was or that was not assessed as described herein can be treated using a composition that includes live Phascolarctobacterium. For example, an obese or over weight human that was not assessed for an elevated level of gut Phascolarctobacterium, a reduced level of gut Veillonella, and/or a reduced level of gut Dialister can be administered, or instructed to self-administer, a composition containing live Phascolarctobacterium.

A composition containing live Phascolarctobacterium for use as described herein can include any appropriate amount of live Phascolarctobacterium. For example, a composition containing live Phascolarctobacterium can include from about 10³ to about 10¹¹ live Phascolarctobacterium. Other ingredients that can be included within a composition containing live Phascolarctobacterium include, without limitation, carbohydrate based bulking agents for capsule dosage form, conventional excipients for tablet dosage form, carbohydrate based bulking agents and/or cryo-lyoprotectants for freeze-dried dosage form, and flavor enhancers, sweeteners, and/or viscosity enhancers for liquid dosage form.

In some cases, a composition containing live Phascolarctobacterium can be administered orally as a probiotic formulation or can be administered as a fecal transplantation formulation.

When treating an obese or over weight mammal (e.g., a human identified as being responsive to an obesity and/or weight loss treatment as described herein) using a composition containing live Phascolarctobacterium, that composition can be administered to that mammal at an appropriate frequency and for an appropriate duration. For example, an obese or over weight human can be administered a composition containing live Phascolarctobacterium from about once a month to about three times a day (e.g., once a month, twice a month, three times a month, once a week, twice a week, three times a week, once a day, twice a day, three times a day, or any range there between). In some cases, an obese or over weight human can be administered a composition containing live Phascolarctobacterium for a duration ranging from about one day to about one year (e.g., about one day, about three days, about one week, about two weeks, about three weeks, about one month, about two months, about three months, about six months, about nine months, about one year, or any range there between. In some cases, an obese or over weight human can be administered a composition containing live Phascolarctobacterium at an appropriate frequency (e.g., once daily, weekly, or monthly) until a desired amount of weight is lost (e.g., until 5 percent, 10 percent, 15 percent, or more of the mammal's pre-treatment weight is lost).

In some cases, an obese or over weight mammal such as an obese or over weight human having a gut microbiota that was or that was not assessed as described herein can be treated using a composition that includes one or more microbiota modifying agents having the ability to reduce the number of Veillonella organisms and/or the number Dialister organisms within the mammal's gut. Examples of microbiota modifying agents having the ability to reduce the number of Veillonella organisms and/or the number Dialister organisms within the mammal's gut include, without limitation, antibiotics and phage therapy. In some cases, a composition that includes one or more microbiota modifying agents having the ability to reduce the number of Veillonella organisms and/or the number Dialister organisms within the mammal's gut can be formulated in a manner such that administration of the composition to a mammal does not reduce the number of Phascolarctobacterium organisms within the mammal's gut. Examples of such compositions included, without limitation, antibiotics and strain specific bacteriophage. In some cases, a composition that includes one or more microbiota modifying agents having the ability to reduce the number of Veillonella organisms and/or the number Dialister organisms within the mammal's gut can be administered to a mammal in a manner that results in a reduction in the number of gut Veillonella organisms and/or the number of gut Dialister organisms in addition to a reduction in the number of gut Phascolarctobacterium organisms. In such cases, a composition containing live Phascolarctobacterium can be administered to the mammal to restore or increase the number of gut Phascolarctobacterium organisms.

A composition containing one or more microbiota modifying agents having the ability to reduce the number of Veillonella organisms and/or the number Dialister organisms within the mammal's gut can include any appropriate amount of the one or more microbiota modifying agents. For example, a composition containing one or more microbiota modifying agents can include from about oneto about five of the one or more microbiota modifying agents having the ability to reduce the number of Veillonella organisms and/or the number Dialister organisms within the mammal's gut.

In some cases, a composition containing one or more microbiota modifying agents having the ability to reduce the number of Veillonella organisms and/or the number Dialister organisms within the mammal's gut can be administered orally.

When treating an obese or over weight mammal (e.g., a human identified as being responsive to an obesity and/or weight loss treatment as described herein) using a composition containing one or more microbiota modifying agents having the ability to reduce the number of Veillonella organisms and/or the number Dialister organisms within the mammal's gut, that composition can be administered to that mammal at an appropriate frequency and for an appropriate duration. For example, an obese or over weight human can be administered a composition containing one or more microbiota modifying agents having the ability to reduce the number of Veillonella organisms and/or the number Dialister organisms within the mammal's gut from about once a month to about three times a day (e.g., about once a month, about twice a month, about three times a month, about once a week, about twice a week, about three times a week, about once a day, about twice a day, about three times a day, or any range there between). In some cases, an obese or over weight human can be administered a composition containing one or more microbiota modifying agents having the ability to reduce the number of Veillonella organisms and/or the number Dialister organisms within the mammal's gut for a duration ranging from about one day to about one month (e.g., about one day, about two days, about three days, about five days, about one week, about two weeks, about three weeks, about one month, or any range there between). In some cases, an obese or over weight human can be administered a composition containing one or more microbiota modifying agents having the ability to reduce the number of Veillonella organisms and/or the number Dialister organisms within the mammal's gut at an appropriate frequency (e.g., once daily, weekly, or monthly) until a desired amount of weight is lost (e.g., until 5 percent, 10 percent, 15 percent, or more of the mammal's pre-treatment weight is lost).

The invention will be further described in the following examples, which do not limit the scope of the invention described in the claims.

### EXAMPLES

### Example 1

### Individualized Responses to Lifestyle Interventions Can Be Predicted By Gut Microbiota

The following was performed to determine if baseline differences in the gut microbiota are predictive of weight loss after a lifestyle intervention program in overweight and obese individuals.

### Methods

Twenty-seven obese and overweight individuals were recruited to participate in a lifestyle intervention program that included cognitive/behavioral therapy, a nutritional intervention, and a physical activity intervention. The nutritional intervention involved a volumetric approach that included unlimited fruits and vegetables and lower energy density foods with greater nutrient density. The physical activity intervention involved walking at least 10,000 steps per day. Subjects were followed every week during the first 3 months, every 2 weeks during the next month, and monthly thereafter.

Height and weight measurements were obtained at baseline, 3 months, and 6 months after initiation of the intervention program. Percent of baseline weight loss also was calculated, and a 5% weight loss was defined as success. Fecal stool samples were collected at baseline, 3 months, and 6 months. The V4 variable region of bacterial 16S rRNA was amplified from stool DNA and sequenced with the MiSeq Illumina platform. Data analysis was done using Quantitative Insights into Microbial Ecology (QIIME 1.9.1) pipeline. The linear discriminant analysis (LDA) effect size (LEfSe) method was performed to identify significantly discriminative bacteria between subjects who failed or succeeded in achieving weight loss. An alpha of 0.05 and LDA threshold of >2.0 were used.

### Results

Three overweight and 24 obese individuals were included in the analysis. Mean age was 54 years (95% CI: 50.7, 57.1) and 78% were female. Average weight at baseline was 96.0 kg (95% CI: 90.4, 101.4), and average BMI at baseline was 34.1 kg/m2 (95% CI: 32.8, 35.4). At 3 months, subjects achieved a mean weight loss of 3.71 kg (95% CI: 2.31, 5.11) and a mean reduction in BMI of 0.80 (95% CI: 0.38, 1.57), with 9 subjects (33%) achieving at least a 5% weight loss. Eight of these subjects (89%) maintained a 5% weight loss at 6 months. Gut microbiota analysis showed no significant changes in alpha diversity or beta diversity after 3 months or 6 months.

Subjects who achieved a 5% weight loss had baseline microbiota significantly enriched with Phascolarctobacterium, Corynebacterium, Cerasicoccaceae family, Oxalobacter, and Turicibacteraceae family bacteria. Those who failed to achieve a 5% weight loss had microbiota enriched with Veillonella and Pasteurellaceae family bacteria. Interestingly, both Phascolarctobacterium and Veillonella are members of the Veillonellaceae family and were the most discriminant taxa for each group, respectively, suggesting a potential nutrient niche competition in different states.

These results demonstrate that the elevated levels of Phascolarctobacterium members within a human's gut microbiota can be used to identify humans capable of experiencing successful weight loss during a weight loss treatment (e.g., a comprehensive lifestyle intervention program). These results also demonstrate that the elevated levels of Veillonella members within a human's gut microbiota can be used to identify humans capable of experiencing unsuccessful weight loss (e.g., less than 5 percent weigh loss) during a weight loss treatment (e.g., a comprehensive lifestyle intervention program).

### Example 2

### Gut Microbial Carbohydrate Metabolism Hinders Weight Loss in Overweight Adults Undergoing Lifestyle Intervention with a Volumetric Diet

### Patient Selection

Patients were recruited from the Mayo Clinic Obesity Treatment Research Program. Adults aged 18-65 years with a BMI of 27-39.9 kg/m² and able to provide informed consent were included in the study. The exclusion criteria included health problems that prevented individuals from engaging in physical activity, previous surgeries for managing obesity (bariatric procedures, gastric bypass surgery), concurrent participation in another weight loss program, and use of weight loss medications with the previous 30 days. For the microbiome analysis arm of the study, participants with any use of antibiotics within the previous 30 days were also excluded.

### Study Interventions

The Mayo Clinic Obesity Treatment Research Program is a 12-month comprehensive lifestyle intervention program. During the first 3 months, participants were followed through weekly one hour sessions, biweekly in the fourth month, and monthly thereafter until 12 months. To minimize the impact of participant non-compliance on the results, the first 3 months was selected as the timeframe of the study. The nutritional intervention involved a volumetric approach that included larger amounts of fruits, vegetables and low energy density foods with lesser intake of foods with greater nutrient density. The goal was to reduce energy intake while achieving a high food intake volume.

The physical activity intervention involved recommendations to walk at least 10,000 steps per day or its equivalent. Physical activity was monitored using a pedometer with 7-day memory. Patients were instructed to wear the pedometer every day and review their step count data to assess progress towards the goal.

The behavioral intervention was given in weekly group sessions and included elements such as self-monitoring, managing expectations, goal setting, stimulus control, stress reduction, problem solving, social support, cognitive restructuring, and relapse prevention. The general outline of the sessions was based upon the Look AHEAD protocol (Look et al., Obesity (Silver Spring), 14(5):737-752 (2006)).

### Outcome Measures and Data Processing

Clinical, biochemical, and demographic information was collected from patients at baseline and after 3 months including age, sex, race, weight, height, BMI, smoking status, hypertension, pre-diabetes, type 2 diabetes, fasting blood glucose, high-density lipoprotein levels, low-density lipoprotein levels, and triglyceride levels. The percent weight loss after 3 months was calculated based on the participant's baseline body weight. A 5% or greater weight loss after 3 months was defined as success. Fecal stool samples were collected at baseline and after 3 months.

DNA isolation from stool samples was performed using the Mo Bio PowerSoil® DNA Isolation Kit (Mo Bio Laboratories; Carlsbad, Calif.) following bead beating. The V4 variable region of bacterial 16S rRNA was amplified from stool DNA and sequenced with the MiSeq Illumina platform. Compositional and diversity data analysis was done using Quantitative Insights into Microbial Ecology (QIIME 1.9.1) pipeline (Caporaso et al., Nat. Methods, 7(5):335-336 (2010)).

Predictive functional profiling from 16 rRNA was performed using the Phylogenetic Investigation of Communities by Reconstruction of Unobserved States (PICRUSt) pipeline (Langille et al., Nat. Biotechnol., 31(9):814-821 (2013)). Gene content was predicted against the following databases: the Kyoto Encyclopedia of Genes and Genomes (KEGG) database (Kanehisa et al., Nucleic Acids Res., 28(1):27-30 (2000)), the Clusters of Orthologous Groups (COG) database (Tatusov et al., Nucleic Acids Res., 28(1):33-36 (2000)), and the Carbohydrate-Active Enzymes (CAZy) database (Lombard et al., Nucleic Acids Res., 42(Database issue):D490-495 (2014)). The linear discriminant analysis (LDA) effect size (LEfSe) method (Segata et al., Genome Biol., 12(6):R60 (2011)) was performed to identify predictive compositional and functional biomarkers for weight loss. An alpha of 0.05 and a LDA threshold of >2.0 were used.

### Statistical Analysis

Statistical analysis was performed using JMP Pro 12 software (SAS Institute; Cary, N.C.) and the QIIME pipeline. Two-sided Wilcoxon rank-sum tests were used to measure differences in bacterial composition and alpha diversity, and Permanova was used for beta diversity. A P value of 0.05 or less was considered statistically significant for all tests.

### Results

### Clinical Features Do Not Predict Response to Lifestyle Intervention

Forty-seven participants volunteered for the study. Twenty-six of 47 participants (55%) were included in the analysis, and 21 participants were excluded for the following reasons: did not return at least one of their stool samples (16), did not return their stool samples on time, hence not representative of their baseline gut microbiota (3), insufficient stool sample for analysis (1), or dropped from the study (1).

Of the 26 participants included in the analysis, 4 were overweight and 22 were obese. The mean age of all participants was 53.5 years (95% CI: 50.3-56.8) with 81% of participants being female. Average weight at baseline was 95.7 kg (95% CI: 90.0-101.4), and average BMI at baseline was 34.1 kg/m² (95% CI: 32.6-35.8). At 3 months, the mean weight loss was 3.71 kg (95% CI: 2.31-5.11), and the mean reduction in BMI was 1.27 (95% CI: 0.69-1.87), with 9 subjects (35%) achieving at least a 5% weight loss in 3 months. Participants who lost at least 5% of their weight lost an average of 7.89 kg (95% CI: 6.46-9.32), and those who lost less than 5% of their baseline weight lost an average of 1.51 kg (95% CI: 0.52-2.49). After 6 months, a total of 8 of 9 participants who lost at least 5% of their baseline weight at 3 months, maintained the weight loss, while an additional 2 participants achieved at least 5% weight loss. A comparison of baseline clinical, biochemical, and demographic characteristics between subjects at 3 months is shown in Table 1. There were no significant differences in these variables at baseline between the success and failure groups. There also were no associations with success at 3 months.

TABLE 1
Distribution of baseline demographic and clinical characteristics.

| | ≥5% weight loss | | <5% weight loss | | Univariate OR | | |
|---|---|---|---|---|---|---|---|
| | (n = 9) | | (n = 17) | | (95% CI) | | P value |
| Age | 51.1 | (43.1-59.1) | 54.8 | (51.5-58.2) | 0.94 | (0.84-1.04) | 0.25 |
| Sex, female (%) | 8 | (89) | 13 | (76) | 2.46 | (0.29-52.81) | 0.63 |
| Race (%) | White: 9 | | White: 16 (94) | | 0 | | 1.00 |
| | (100) | | Asian: 1 (6) | | | | |
| Weight, kg | 99.0 | (86.9-111.1) | 94.0 | (87.1-100.9) | 1.03 | (0.97-1.10) | 0.39 |
| Height, cm | 169.5 | (162.5-176.5) | 166.1 | (161.2-171.2) | 1.04 | (0.95-1.14) | 0.38 |
| BMI | 34.3 | (31.4-37.3) | 34.0 | (32.1-35.9) | 1.03 | (0.82-1.31) | 0.81 |
| Smoking (%) | 1 | (11) | 1 | (6) | 0.50 | (0.03-9.08) | 1.00 |
| Hypertension | 4 | (44) | 5 | (29) | 1.92 | (0.35-10.70) | 0.67 |
| Fasting glucose | 94 | (89-100) | 103 | (96-110) | 0.91 | (0.80-1.00) | 0.05 |
| Pre-diabetes (%) | 4 | (44) | 9 | (53) | 0.71 | (0.13-3.62) | 0.68 |
| Type 2 diabetes (%) | 0 | | 2 | (12) | 0 | | 0.53 |
| HDL | 53.0 | (40.2-65.8) | 58.5 | (50.19-66.7) | 0.98 | (0.92-1.03) | 0.40 |
| LDL | 118.6 | (100.5-136.6) | 125.0 | (104.3-145.8) | 0.99 | (0.97-1.02) | 0.64 |
| Triglycerides | 145.6 | (86.8-204.3) | 170.6 | (127.42-213.8) | 1.00 | (0.98-1.01) | 0.43 |

*BMI: body mass index; HDL: high density lipoprotein; LDL: low-density lipoprotein; OR: odds ratio

Compositional Differences Within the Veillonellaceae Family at Baseline were Associated with ≥5% Weight Loss

Microbial compositional analysis using LEfSe identified two members of gut microbiota that were significantly different between the groups.

Phascolarctobacterium was significantly increased at baseline in subjects that lost at least a 5% weight loss after 3 months (LDA 2.09, P=0.008), and Dialister was significantly increased in those with less than 5% weight loss (LDA −2.07, P=0.03).
A Gut Microbiota with Increased Capacity for Carbohydrate Metabolism at Baseline was Associated with Failure to Lose ≥5% of Baseline Weight

Gut microbiota functionality was imputed from compositional data using PICRUSt. The abundance of predicted genes was then analyzed through LEfSe to identify predictors of weight loss. A transposase (COG3328) was identified that was predictive of success. Six carbohydrate-active enzymes and binding proteins that were predictive of less than 5% weight loss after 3 months were also identified (Table 2). This included two glycoside hydrolase families (GH67 and GH93), two carbohydrate-binding module families (CBM51 and CBM58), a carbohydrate esterase family (CE6), and a glycosyltransferase family (GT25).

### Table 2. Carbohydrate-Active Enzymes and Proteins Found to be Predictive of Responses to a 3-Month Lifestyle Intervention.

TABLE 2
Carbohydrate-active enzymes and proteins found to be predictive of
responses to a 3-month lifestyle intervention.
Carbohydrate-active enzymes associated with failure to lose 5% of
weight in 3 months

| Reference database | |
|---|---|
| identifier | Function |
| CBM51 | Carbohydrate-binding module: binds blood group A |
| | and B antigens |
| GH67 | Glycoside hydrolase |
| CBM58 | Carbohydrate-binding module: notable member is |
| | SusG, an α-amylase |
| GH93 | Glycoside hydrolase |
| CE6 | Carbohydrate esterase |
| GT25 | Glycosyltransferase |

### Baseline Gut Microbial Diversity is Similar Regardless of Achieved Weight Loss After 3 Months

Analysis of baseline gut microbial diversity between subjects in the two groups revealed no significant differences in alpha diversity (observed OTUs, Shannon index, and Chaol metrics) or (beta diversity (Bray-Curtis dissimilarity, unweighted and weighted UniFrac).

### No Observed Change in Gut Microbial Composition and Diversity After 3-Month Intervention

To determine if weight loss during a lifestyle intervention program led to changes in gut microbiota composition, interval changes in gut microbial composition and diversity were analyzed after 3 months. There were no significant changes in alpha diversity (observed OTUs, Shannon index, and Chaol metrics) or (beta diversity (Bray-Curtis dissimilarity, unweighted and weighted UniFrac) or bacterial composition in either group of patients.

The results provided herein demonstrate that particular compositional and functional bacterial profiles were associated with >5% weight loss responses in overweight and obese adults undergoing a 3-month comprehensive lifestyle intervention program. Increased baseline abundance of Phascolarctobacterium was associated with a weight loss of at least 5%, while increased abundance of Dialister was associated with a weight loss of less than 5%. Interestingly, both genera belong to the Veillonellaceae family, which suggests that compositional shifts within this family may have a role in host energy metabolism. The results provided herein also demonstrate that an increased abundance of several carbohydrate metabolizing enzymes was associated with outcomes. In some cases, the combination of increased microbial carbohydrate-active enzyme pathways and decreased abundance of Phascolarctobacterium in the gut microbiota of obese and overweight individuals can be associated with failure to lose at least 5% weight following a comprehensive lifestyle intervention program.

### Example 3

### Compositional Differences Within the Veillonellaceae Family at Baseline were Associated with at Least 5% Weight Loss

Microbial compositional analysis using LEfSe identified two members of the gut microbiota that were significantly different between the groups.

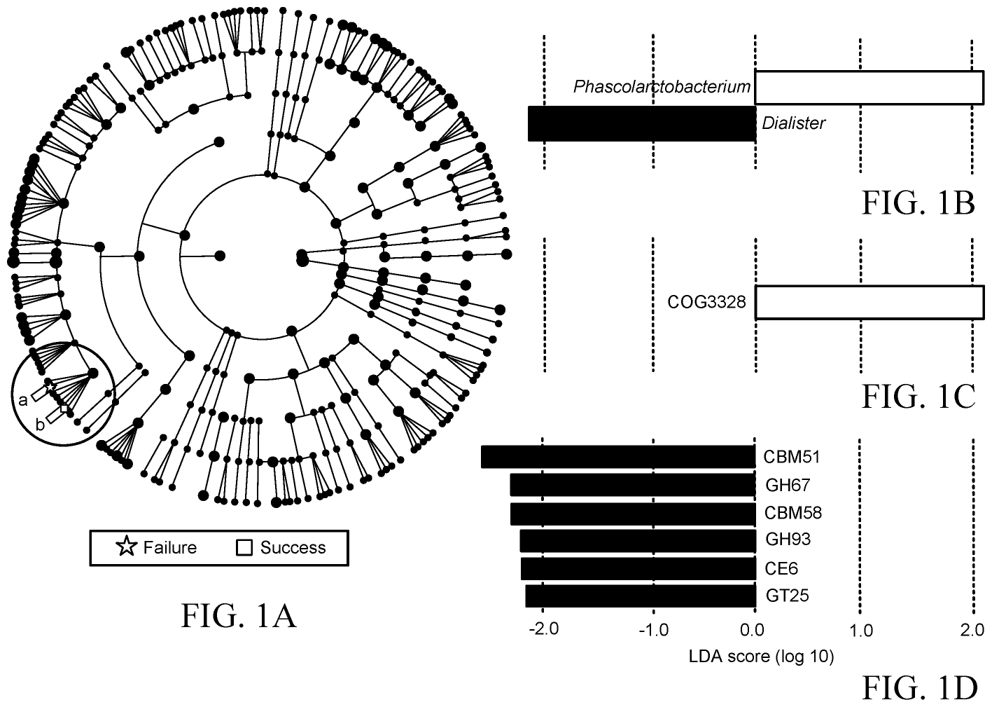

Phascolarctobacterium was significantly increased at baseline in participants who lost at least 5% weight loss after 3 months (LDA score, 2.09; P=0.008) and Dialister was significantly increased in those who achieved less than 5% weight loss (LDA score, −2.07; P=0.03) (FIGS. 1A and 1B).

### Example 4

### A Gut Microbiota with Increased Capacity for Carbohydrate Metabolism at Baseline was Associated with Failure to Lose at Least 5% of Baseline Weight

Gut microbiota functionality was imputed from compositional data using the Phylogenetic Investigation of Communities by Reconstruction of Unobserved States pipeline. The abundance of predicted genes was then analyzed through LEfSe to identify biomarkers of weight loss. A gene encoding a transposase (COG3328) that was predictive of success (FIG. 1C) was identified. In addition, six bacterial genes encoding carbohydrate-active enzymes and binding proteins that were predictive of less than 5% weight loss after 3 months were identified. These included two glycoside hydrolase families (GH67 and GH93), two carbohydrate-binding module families (CBM51 and CBM58), a carbohydrate esterase family (CE6), and a glycosyltransferase family (GT25) (FIG. 1D).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-9. (canceled)

10. A method for treating an overweight or obese mammal, wherein said method comprises treating a mammal identified as having an elevated level of Phascolarctobacterium within the mammal's gut microbiota, a reduced level of Veillonella within the mammal's gut microbiota, or a reduced level of Dialister within the mammal's gut microbiota with an obesity or over weight treatment.

11. The method of claim 10, wherein said mammal is a human.

12. The method of claim 10, wherein said treatment is a diet or physical exercise.

13. A method for increasing the effectiveness of a weight loss treatment, wherein said method comprises administering a composition comprising live Phascolarctobacterium to a mammal, wherein the level of Phascolarctobacterium within said mammal is increased.

14. The method of claim 13, wherein said mammal is a human.

15. The method of claim 13, wherein said weight loss treatment is a diet or physical exercise.

16. The method of claim 13, wherein over 90 percent of the live bacteria of said composition are Phascolarctobacterium.

17. The method of claim 13, wherein over 95 percent of the live bacteria of said composition are Phascolarctobacterium.

18. The method of claim 13, wherein all the live bacteria of said composition are Phascolarctobacterium.

19. A method for increasing the effectiveness of a weight loss treatment, wherein said method comprises administering, to a mammal, a composition comprising a microbiome modifying agent having the ability to reduce the number of Veillonella organisms or Dialister organisms within said mammal, wherein the number of Veillonella organisms or Dialister organisms within said mammal is reduced.

20. The method of claim 19, wherein said mammal is a human.

21. The method of claim 19, wherein said weight loss treatment is a diet or physical exercise.

22. The method of claim 19, wherein said microbiome modifying agent is antibiotics.
